(19) United States
(12) Patent Application Publication
(10) Pub. No.: US 2011/0301544 A1
(43) Pub. Date: Dec. 8, 2011

(54) INTRAVENOUS LINE SUPPORT SLEEVE

(76) Inventor: DAVID M. DIXON, Tucson, AZ (US)

(21) Appl. No.: 13/022,482

(22) Filed: Feb. 7, 2011

Related U.S. Application Data
(63) Continuation-in-part of application No. 11/752,244, filed on May 22, 2007, now abandoned.
(60) Provisional application No. 60/747,915, filed on May 22, 2006.

(51) Int. Cl. A61M 39/00

(52) U.S. Cl. 604/179; 604/174

(57) ABSTRACT

A planar assembly for use as a tubular intravenous line support sleeve comprising a trapezoidal-shaped center portion, an insertion tab extending outwardly from a first side of said center portion, and an insertion slot assembly extending outwardly from a second and opposing side of said center portion.

### CROSS REFERENCE TO RELATED APPLICATIONS

This application is a Continuation In Part application claiming priority from a U.S. Non-Provisional application having Ser. No. 11/752,244 which claimed priority from a U.S. Provisional application having Ser. No. 60/747,915, filed May 22, 2006, the entire contents of which is incorporated herein by reference.

### FIELD OF THE INVENTION

This invention relates to an intravenous line support sleeve, and a method using same.

### BACKGROUND OF THE INVENTION

It is known in the art to administer medicaments to a patient via intravenous infusion. An intravenous catheter is advanced through the back of a patient hand and is removeably disposed into a vein. One or more medicaments are disposed in a fluid reservoir, and that fluid reservoir is interconnected to the catheter using an intravenous line comprising a flexible tubular member.

To prevent inadvertent removal of the intravenous catheter from the vein, prior art methods include securing a portion of the intravenous line to patient's forearm using tape. Subsequent removal of that tape can cause tearing of, and/or other damage to, the patient's skin.

This invention relates to an apparatus to prevent tears or damage to the skin caused by various adhesive tapes after securing one or more intravenous lines to a patient's forearm.

### SUMMARY OF THE INVENTION

A planar assembly for use as a tubular intravenous line support sleeve is presented. The planar assembly comprises a trapezoidal-shaped center portion, an insertion tab extending outwardly from a first side of said center portion, and an insertion slot assembly extending outwardly from a second and opposing side of said center portion.

A method using the planar assembly is presented. The method includes wrapping the planar assembly around a patient's forearm, and inserting the insertion tab into one of the plurality of slots formed in the insertion slot assembly to form a tubular intravenous line support sleeve comprising the shape of a truncated cone, wherein the tubular intravenous line support sleeve comprises a free floating platform disposed around but not attached to the patient's forearm to be used as an attachment surface to which securing tape can be applied rather than attaching that securing tape to the patient's skin.

### BRIEF DESCRIPTION OF THE DRAWINGS

The invention will be better understood from a reading of the following detailed description taken in conjunction with the drawings in which like reference designators are used to designate like elements, and in which:

FIG. 1A is a perspective view showing a first surface of a first embodiment Applicant's trapezoid-shaped member;

FIG. 1B is a perspective view showing a second surface of the trapezoid-shaped member of FIG. 1A, wherein a flexible foam member is disposed on that second surface;

FIG. 1C is a perspective view showing a first surface of a second embodiment Applicant's trapezoid-shaped member, wherein that trapezoid-shaped member is formed to include a plurality of apertures extending therethrough;

FIG. 2 shows the sides of the trapezoid-shaped member of FIG. 1A being aligned to defined a tubular member;

FIG. 3A shows one embodiment of the attachment means used to convert Applicant's trapezoid-shaped member into a tubular member;

FIG. 3B shows a second embodiment of the attachment means used to convert Applicant's trapezoid-shaped member into a tubular member;

FIG. 3C shows the attachment means of FIG. 3B releaseably attached to one another to form a tubular member;

FIG. 4 shows a third embodiment of the attachment means used to convert Applicant's trapezoid-shaped member into a tubular member;

FIG. 5 shows the attachment means of FIG. 4 releaseably attached to one another to form a tubular member;

FIG. 6A shows a plurality of Applicant's fixturing assemblies disposed on the trapezoid-shaped member of FIG. 1A;

FIG. 6B shows a plurality of Applicant's fixturing assemblies disposed on the trapezoid-shaped member of FIG. 1C;

FIG. 7 shows the fixturing assemblies of FIG. 6A in a receiving configuration; and

FIG. 8A shows a first view of a first embodiment of Applicant's intravenous line support sleeve removeably disposed around a patient's forearm with an intravenous line releaseably attached thereto;

FIG. 8B shows a second view of the first embodiment of Applicant's intravenous line support sleeve removeably disposed around a patient's forearm with an intravenous line releaseably attached thereto;

FIG. 9A shows Applicant's planar assembly for use as a tubular intravenous line support sleeve;

FIG. 9B shows a second embodiment of the planar assembly of FIG. 9A;

FIG. 9C shows additional elements of the planar assemblies of FIGS. 9A and 9B;

FIG. 10 shows a planar assembly of either FIG. 9A or FIG. 9 disposed beneath a patient's forearm;

FIG. 11 shows Applicant's tubular intravenous line support sleeve disposed around a patient's forearm; and

FIG. 12 shows Applicant's tubular intravenous line support sleeve formed using a planar assembly of FIG. 9A or a planar assembly of FIG. 9B disposed around a patient's forearm with an intravenous line releaseably attached thereto.

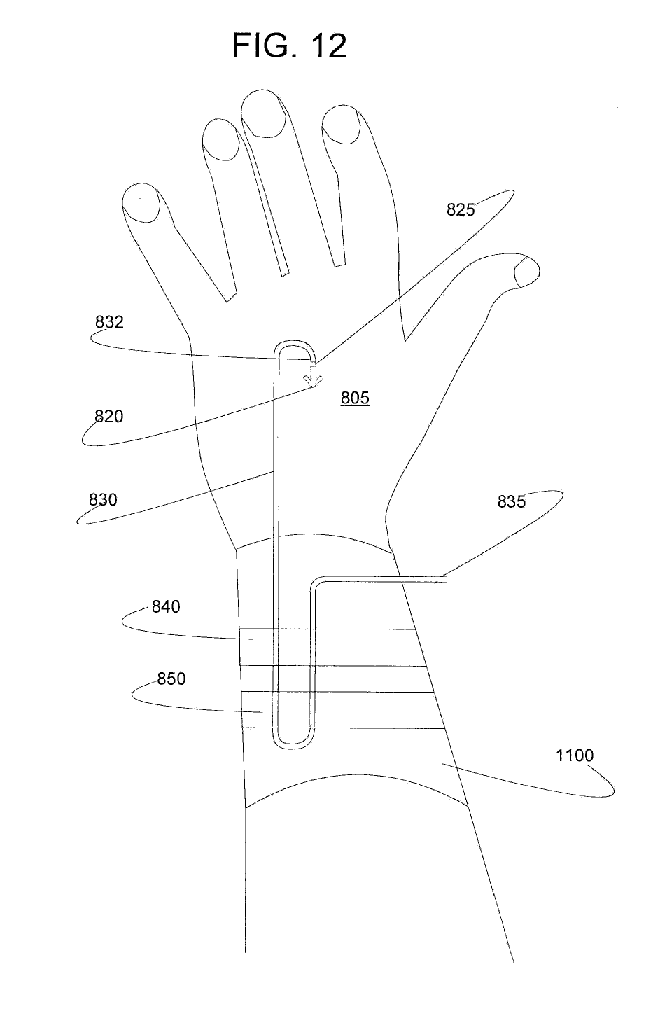

### DETAILED DESCRIPTION OF THE PREFERRED EMBODIMENTS

This invention is described in preferred embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

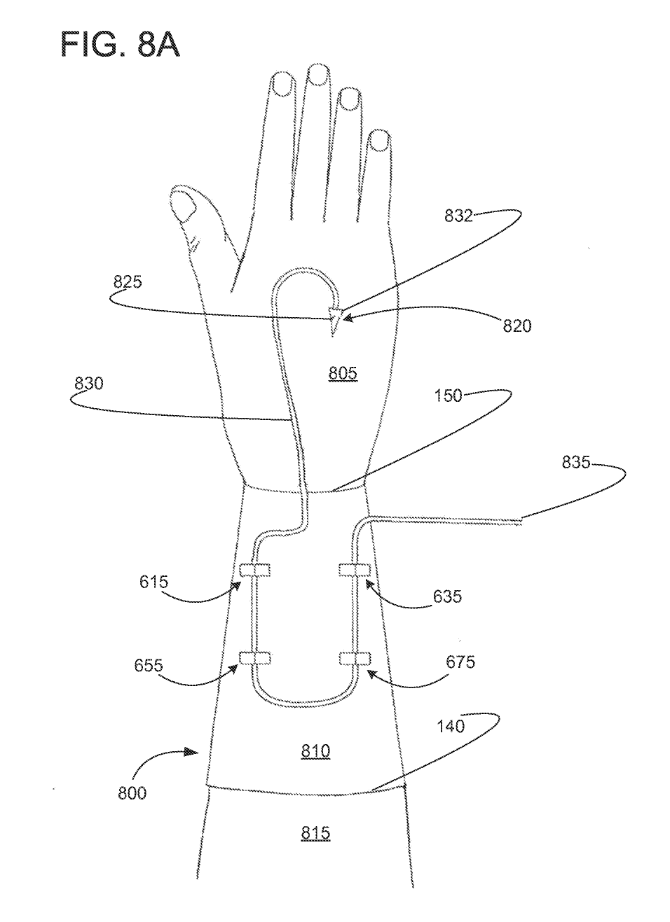

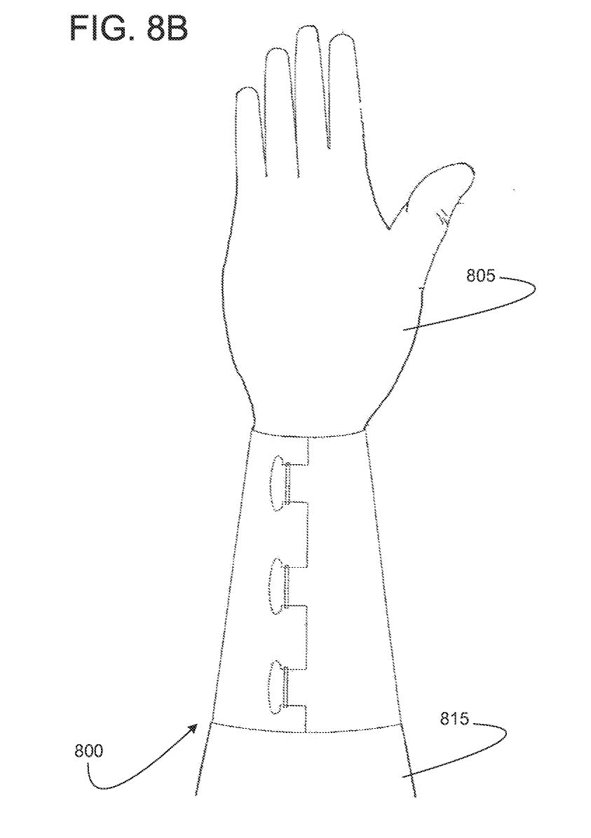

FIG. 8 shows Applicant's intravenous line support sleeve 800 removeably disposed around a patient's forearm 815. Applicant's intravenous line support sleeve functions, inter alia, as a forearm protector. In the illustrated embodiment if FIG. 8, intravenous line support sleeve 800 comprises a plurality of intravenous line fixturing assemblies 615, 635, 655, and 675. Intravenous line support sleeve member 800 comprises first end 140 and opposing second end 150. In certain embodiments, Applicant's intravenous line support sleeve may comprise padding disposed on all or a portion of the surface in contact with a patient's skin.

In the illustrated embodiment of FIG. 8, intravenous catheter 820 has been advanced through the skin and removeably disposed in a vein disposed within a patient's hand 805. A portion 825 of that catheter remains external to hand 805. A first end 832 of intravenous line 830 is interconnected with catheter portion 825. A second end 835 of intravenous line 830 is interconnected with a fluid reservoir, wherein that fluid reservoir contains an infusible liquid formulation comprising a fluid medium, such as and without limitation water, saline, Ringer's Lactate, and the like, in optional combination with one or more medicaments disposed in the fluid medium. By “intravenous line,” Applicant means a flexible tubular member used to interconnect a fluid reservoir and an intravenous catheter.

In certain embodiments, Applicant's intravenous line support sleeve comprises one or more of paper, cloth, plastic, and combinations thereof. In the illustrated embodiment of FIG. 8, no portion of Applicant's intravenous line support sleeve 800 is secured to, or attached in any way to, the patient's skin. In essence, intravenous line support sleeve 800 “floats” on the forearm. Applicant's intravenous line support sleeve provides a substitute skin surface to one or more secure I.V. lines rather than attaching those line directed to the patient's often compromised skin.

The size and shape of the Applicant's intravenous line support sleeve prevents movement along the length of the patient's arm so the IV line(s) remain secured to the patient while at the same time protecting the patient from an I.V. “tug” injury.

Applicant's intravenous line support sleeve is not intended as a wound dressing. Rather, Applicant's device comprises a free floating platform solely intended to be an attachment surface to which, for example and without limitation, securing tape is applied rather than attaching that tape to the patient's forearm. As those skilled in the art will appreciate, a patient's forearm may not tolerate repeated application of, and removal of, securing tapes without damage to the skin.

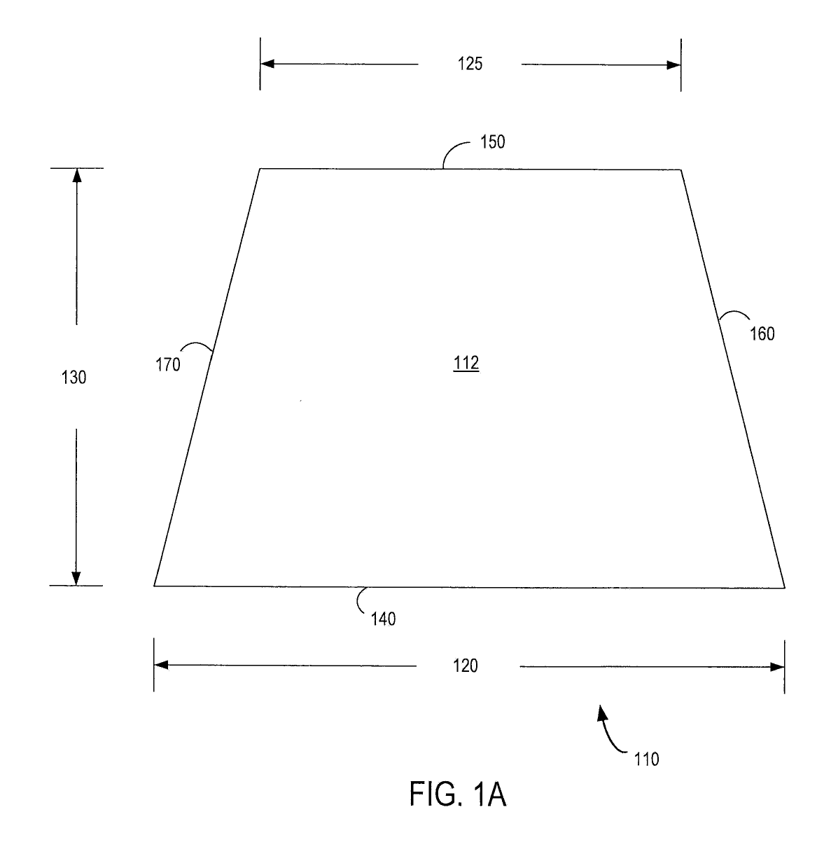

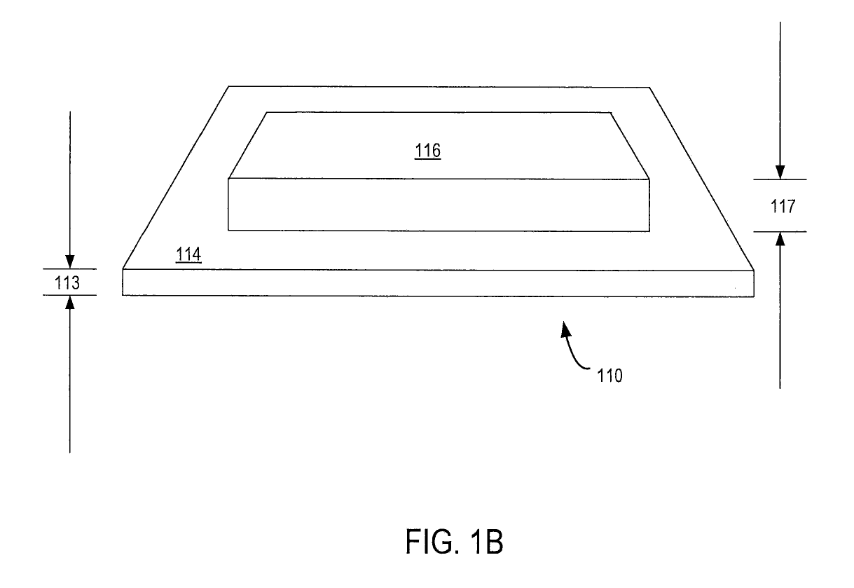

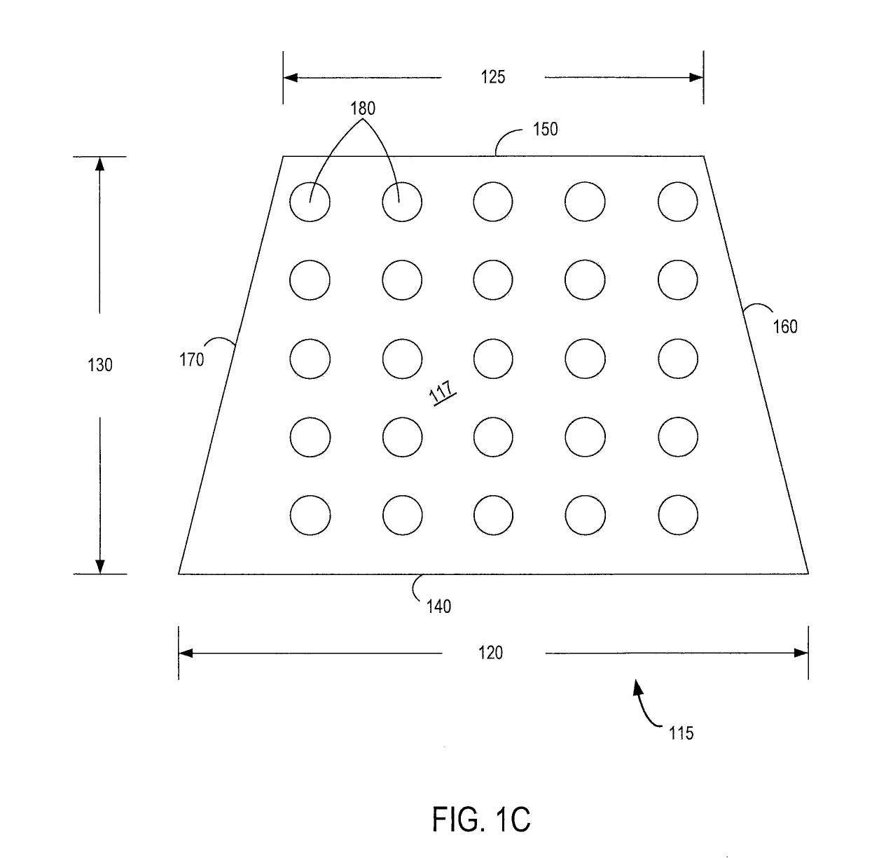

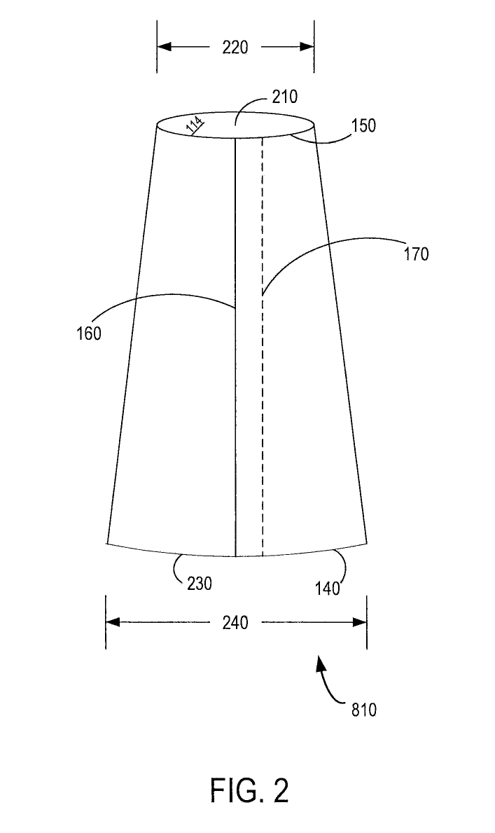

Referring now to FIGS. 1A and 8, in certain embodiments Applicant's intravenous line support sleeve 800 comprises a trapezoid-shaped member 110 comprising a first surface 112, second surface 114 (FIGS. 1B, 2), first end 140 and an opposing second end 150, a first side 160 and an opposing second side 170. Member 110 further comprises a first width 120 along first end 140, second width 125 along second end 150, length 130, and thickness 113 (FIG. 1B).

In certain embodiments, width 120 is between about 20 centimeters and about 28 centimeters. In certain embodiments, width 125 is between about 17 centimeters and about 20 centimeters. In certain embodiments, length 130 is between about 11 centimeters and about 18 centimeters. In certain embodiments, thickness 113 is between about 0.15 millimeters and about 2.0 millimeters.

Referring now to FIG. 1B, in certain embodiments flexible foam member 116 is disposed on surface 114 of member 110. In these embodiments, foam member 116 comprises a length less than length 130, and a width less than width 150. Foam member 116 further comprises thickness 117. In certain embodiments, thickness 117 is greater than thickness 113.

Referring now to FIGS. 1C and 8, in certain embodiments Applicant's intravenous line support sleeve 800 comprises a trapezoid-shaped member 115 formed to include a plurality of apertures 180 extending therethrough, and further comprising a first surface 117, first end 140 and an opposing second end 150, a first side 160 and an opposing second side 170. Member 110 further comprises a first width 120 along first end 140, second width 125 along second end 150, length 130, and thickness 113.

Apertures 180 facilitate contact between the patient's forearm 815 (FIG. 8) and ambient air, thereby maintaining a normal body temperature for that covered forearm. In addition, member 115 is lighter in weight than is member 110.

In certain embodiments, intravenous line support sleeve 800 comprises a trapezoid-shaped member 115 in combination with flexible foam member 116. In these embodiments, apertures 180 extend through both member 115 and foam member 115.

Referring now to FIGS. 2 and 8, tubular member 810 is formed by overlapping sides 160 and 170 of trapezoid-shaped member 110 or 115 to form tubular member 810. In the illustrated embodiment of FIG. 2, tubular member 810 comprises the shape of a truncated cone, wherein the diameter 220 of first open end 210 is smaller than diameter 230 of second open end 240. In other embodiments, tubular member comprises a cylindrical shape, wherein diameter 220 is substantially the same as diameter 240. By “substantially the same,” Applicant means diameters 220 and 230 differ by ten percent or less.

Diameters 220 and 240 are each independently adjustable. In addition, the height 130 of members 110 and/or 115 can be decreased by removing a portion of member 110/115 adjacent to end 140 and/or end 150. Therefore, the dimensions of tubular member 810 can be adjusted such that tubular member remains in close and comfortable contact with the patient's forearm 815.

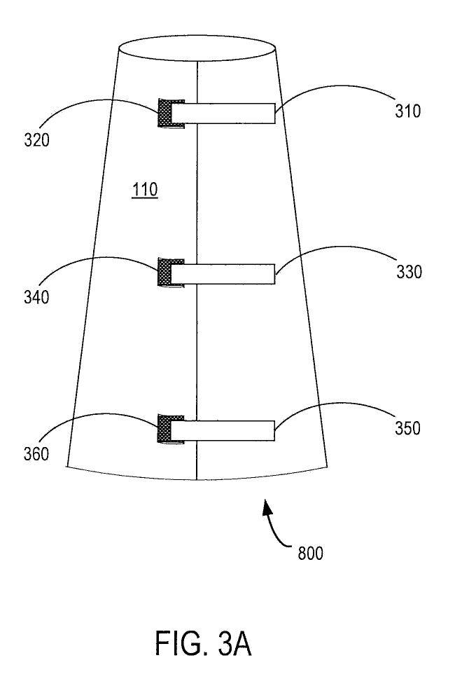

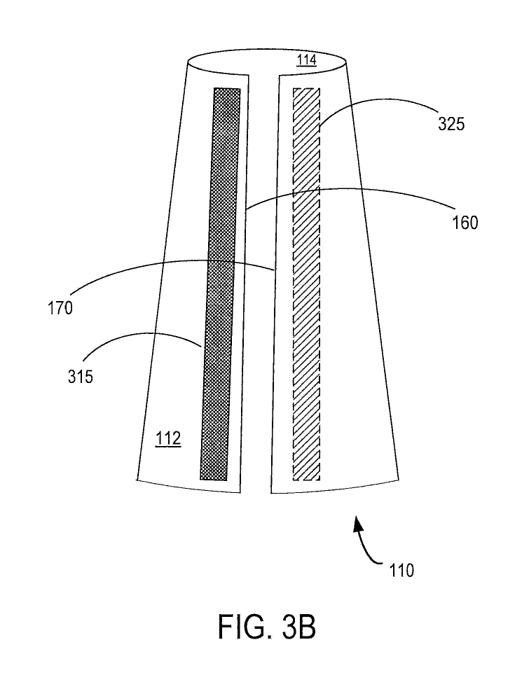

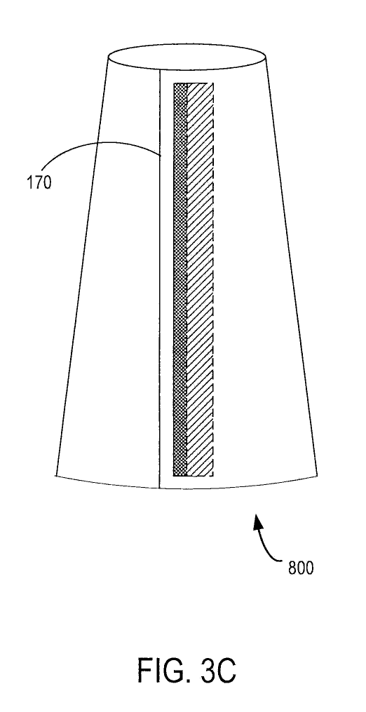

Referring to FIGS. 2 and 3, member 110/115 further comprises a plurality of first attachment means and second attachment means to releaseably secure the member 110/115 in the tubular configuration shown in FIGS. 2, 3A, 3C, 5, and 8. In certain embodiments, member 110/115 comprises a plurality of first attachment means disposed along side 160, and a plurality of second attachment means disposed along side 170, such that member 110/115 can be releaseably configured into tubular member 810.

In the illustrated embodiment of FIG. 3A, member 110 comprises a plurality of hook and loop fastener assemblies, such as hook and loop fastener assemblies 310/320, 330/340, and 350/360. In the illustrated embodiment of FIG. 3, flexible member 310 comprises a proximal end attached to side 160 of member 110, a distal end extending outwardly from side 160, and a fastening means, such as fabric comprising a plurality of hook-type fasteners, disposed on the interior surface of that distal end such that the plurality of hook-type fasteners contact, and releaseably attach to, the fastener-receiving means 320. Hook and loop fastener assemblies 330/340, and 350/360, operate similarly. In other embodiments, fastener assemblies 310/320, 330/340, and 350/360, comprise snaps disposed on the distal ends of flexible members 310, 330, and 350, wherein those snaps are received by, and releaseably attach to, snap-receiving devices 320, 340, and 360.

In the illustrated embodiment of FIGS. 3B and 3C, a plurality of hook-type fasteners 315 are disposed adjacent side 160 on first surface 112 of member 110, and a plurality of loop-type fasteners 325 are disposed adjacent side 170 on second surface 114 of member 110. Releaseably attaching plurality of hook-type fasteners 315 to plurality of loop-type fasteners 325 forms tubular member 800.

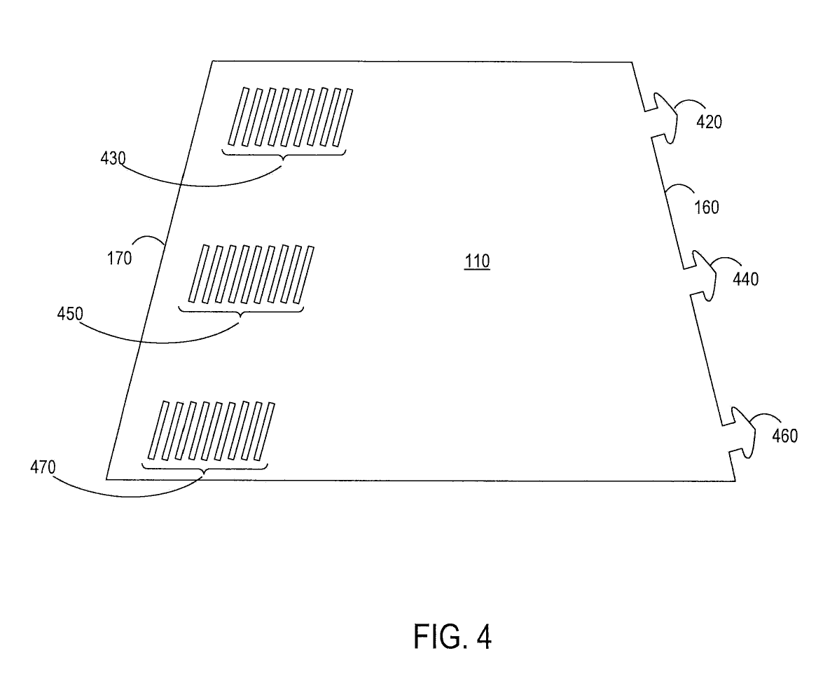

In certain embodiments each first attachment means comprises a tab which can be removeably inserted into a receiving slot. In the illustrated embodiment of FIG. 4, member 110 comprises insertion members 420, 440, and 460, attached to and extending outwardly from side 160. Member 110 is further formed to include adjacent side 170 a first plurality of receiving slots 430, a second plurality of receiving slots 450, and a third plurality of receiving slots 470.

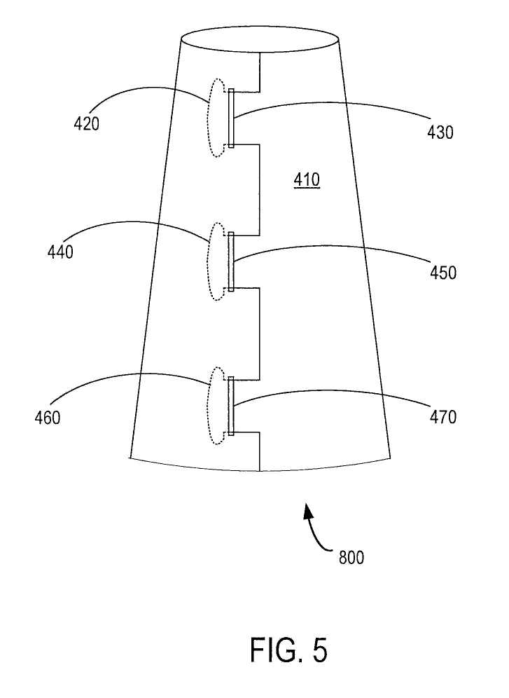

Referring now to FIGS. 4 and 5, removeably disposing insertion member 420 into one of the plurality of receiving slots 430, removeably disposing insertion member 440 into one of the plurality of receiving slots 450, and removeably disposing insertion member 460 into one of the plurality of receiving slots 470, forms the embodiment of tubular member 810 shown in FIG. 5. The choice of which one of receiving slots 430 is used, and the choice of which one of receiving slots 450 is used, and the choice of which one of receiving slots 470 is used, determines the size of diameter 220 (FIG. 2) of first opening 210 (FIG. 2), and the size of diameter 240 (FIG. 2) of second open end 230 (FIG. 2).

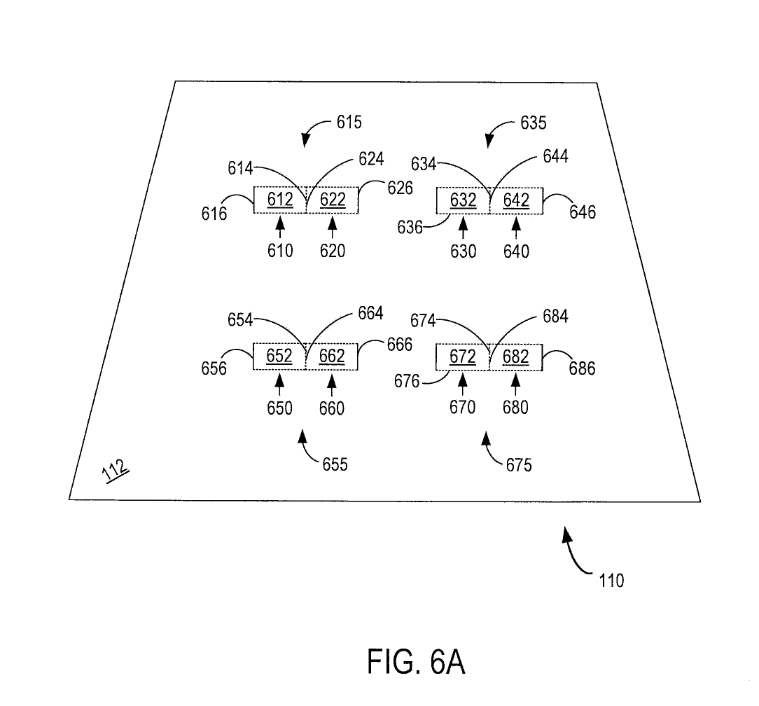

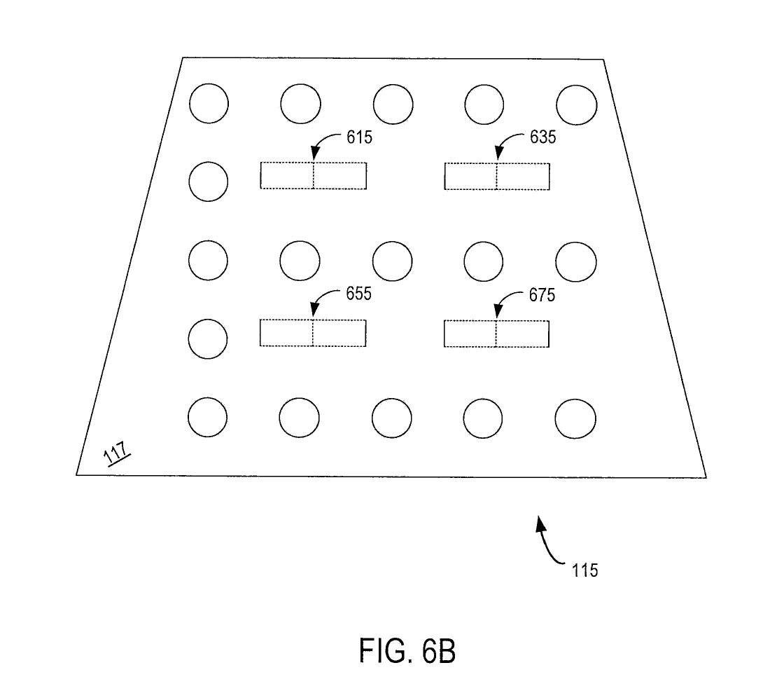

Referring now to FIG. 6A, in certain embodiments member 110 is formed to include intravenous line fixturing assemblies 615, 635, 655, and 675. FIG. 6B shows member 115 formed to comprise fixturing assemblies 615, 635, 655, and 675.

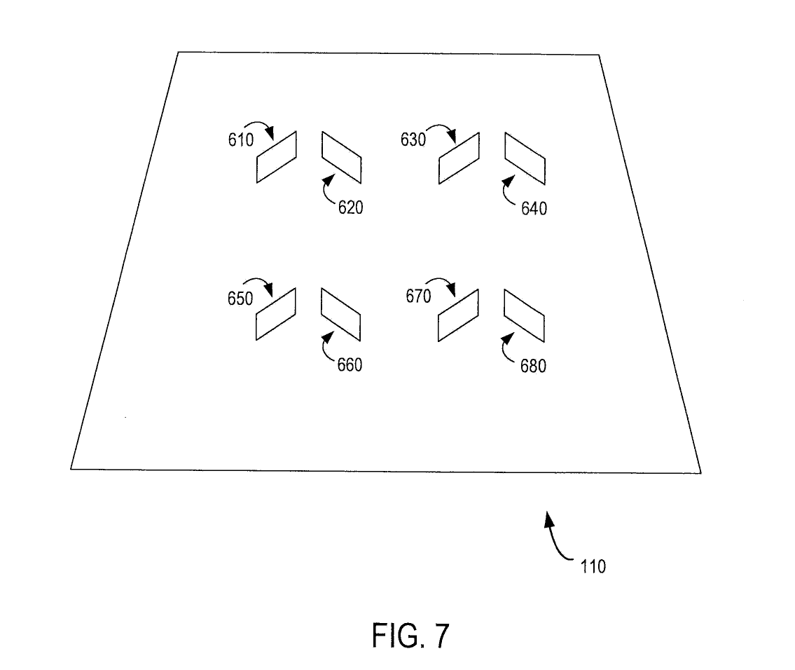

Referring now to FIGS. 6A and 7, fixturing assembly 615 comprises tab 610 and tab 620. Tab 610 comprises rectangular member 612, distal end 614, and proximal end 616. Proximal end 616 is hingedly attached to surface 112 of member 110. In certain embodiments, proximal end 616 is integral with surface 112. Tab 620 comprises rectangular member 622, distal end 624, and proximal end 626. Proximal end 626 is hingedly attached to surface 112 of member 110. In certain embodiments, proximal end 626 is integral with surface 112.

In certain embodiments, member 110 is formed to comprise tabs 610 and 620. In the illustrated embodiment of FIG. 6A, distal end 614 of tab 610 abuts distal end 624 of tab 620. Distal end 614 of tab 610 can be lifted and tab 610 partially rotated about proximal end 616, and distal end 624 of tab 620 can be lifted and tab 620 partially rotated about proximal end 626, to place fixturing assembly 615 into the receiving configuration shown in FIG. 7.

Fixturing assembly 635 comprises tab 630 and tab 640. Tab 630 comprises rectangular member 632, distal end 634, and proximal end 636. Proximal end 636 is hingedly attached to surface 112 of member 110. In certain embodiments, proximal end 636 is integral with surface 112. Tab 640 comprises rectangular member 642, distal end 644, and proximal end 646. Proximal end 646 is hingedly attached to surface 112 of member 110. In certain embodiments, proximal end 646 is integral with surface 112.

In certain embodiments, member 110 is formed to comprise tabs 630 and 640. In the illustrated embodiment of FIG. 6A, distal end 634 of tab 630 abuts distal end 644 of tab 640. Distal end 634 of tab 630 can be lifted and tab 630 partially rotated about proximal end 636, and distal end 644 of tab 640 can be lifted and tab 640 partially rotated about proximal end 646, to place fixturing assembly 635 into the receiving configuration shown in FIG. 7.

Fixturing assembly 655 comprises tab 650 and tab 660. Tab 650 comprises rectangular member 652, distal end 654, and proximal end 656. Proximal end 656 is hingedly attached to surface 112 of member 110. In certain embodiments, proximal end 656 is integral with surface 112. Tab 660 comprises rectangular member 662, distal end 664, and proximal end 666. Proximal end 666 is hingedly attached to surface 112 of member 110. In certain embodiments, proximal end 666 is integral with surface 112.

In certain embodiments, member 110 is formed to comprise tabs 650 and 660. In the illustrated embodiment of FIG. 6A, distal end 654 of tab 650 abuts distal end 664 of tab 660. Distal end 654 of tab 650 can be lifted and tab 650 partially rotated about proximal end 656, and distal end 664 of tab 660 can be lifted and tab 660 partially rotated about proximal end 666, to place fixturing assembly 655 into the receiving configuration shown in FIG. 7.

Fixturing assembly 675 comprises tab 670 and tab 680. Tab 670 comprises rectangular member 672, distal end 674, and proximal end 676. Proximal end 676 is hingedly attached to surface 112 of member 110. In certain embodiments, proximal end 676 is integral with surface 112. Tab 680 comprises rectangular member 682, distal end 684, and proximal end 686. Proximal end 686 is hingedly attached to surface 112 of member 110. In certain embodiments, proximal end 686 is integral with surface 112.

In certain embodiments, member 110 is formed to comprise tabs 670 and 680. In the illustrated embodiment of FIG. 6A, distal end 674 of tab 670 abuts distal end 684 of tab 680. Distal end 674 of tab 670 can be lifted and tab 670 partially rotated about proximal end 676, and distal end 684 of tab 680 can be lifted and tab 680 partially rotated about proximal end 686, to place fixturing assembly 675 into the receiving configuration shown in FIG. 7.

Referring now to FIGS. 7 and 8, intravenous line 830 is first routed as shown in FIG. 8 through fixturing assemblies 615, 636, 655, and 675, while those fixturing assemblies are disposed in the receiving configurations of FIG. 7. Distal ends 614 and 624 of tabs 610 and 620, respectively, are then urged downwardly, and distal ends 634 and 644 of tabs 630 and 640, respectively, are then urged downwardly, and distal ends 654 and 664 of tabs 650 and 660, respectively, are then urged downwardly, and distal ends 674 and 684 of tabs 670 and 680, respectively, are then urged downwardly, to releaseably secure intravenous line 830 to surface 112 of member 110 as shown in FIG. 8.

In the illustrated embodiment if FIG. 8A, intravenous line support sleeve 800 comprises a plurality of intravenous line fixturing assemblies 615, 635, 655, and 675. Intravenous line support sleeve member 800 comprises first end 140 and opposing second end 150. In certain embodiments, Applicant's intravenous line support sleeve may comprise padding disposed on all or a portion of the surface in contact with a patient's skin.

In the illustrated embodiment of FIG. 8, intravenous catheter 820 has been advanced through the skin and removeably disposed in a vein disposed within a patient's hand 805. A portion 825 of that catheter remains external to hand 805. A first end 832 of intravenous line 830 is interconnected with catheter portion 825. A second end 835 of intravenous line 830 is interconnected with a fluid reservoir, wherein that fluid reservoir contains an infusible liquid formulation comprising a fluid medium, such as and without limitation water, saline, Ringer's Lactate, and the like, in optional combination with one or more medicaments disposed in the fluid medium. By “intravenous line,” Applicant means a flexible tubular member used to interconnect a fluid reservoir and an intravenous catheter.

In certain embodiments, Applicant's intravenous line support sleeve comprises one or more of paper, cloth, plastic, and combinations thereof. In the illustrated embodiment of FIG. 8, no portion of Applicant's intravenous line support sleeve 800 is secured to, or attached in any way to, the patient's skin. In essence, intravenous line support sleeve 800 “floats” on the forearm. Applicant's intravenous line support sleeve provides a substitute skin surface to one or more secure I.V. lines rather than attaching those line directed to the patient's often compromised skin.

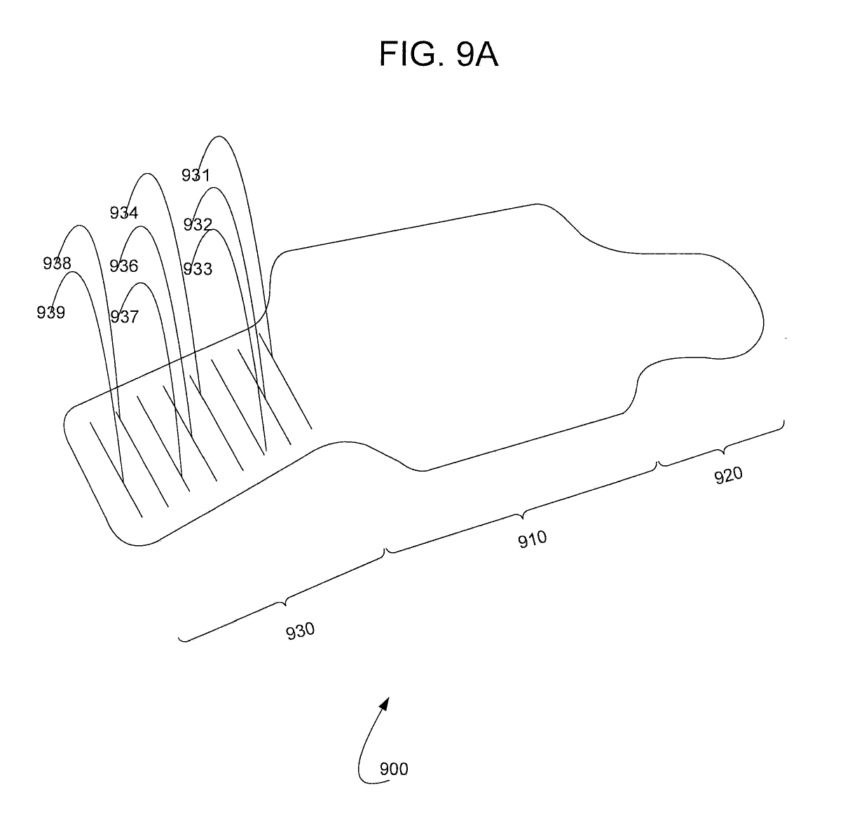

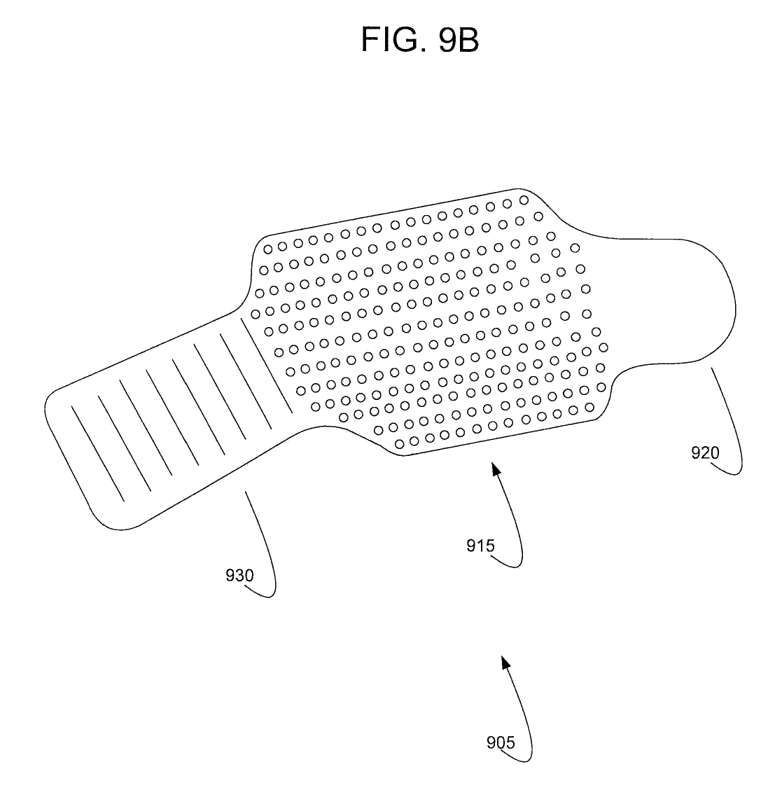

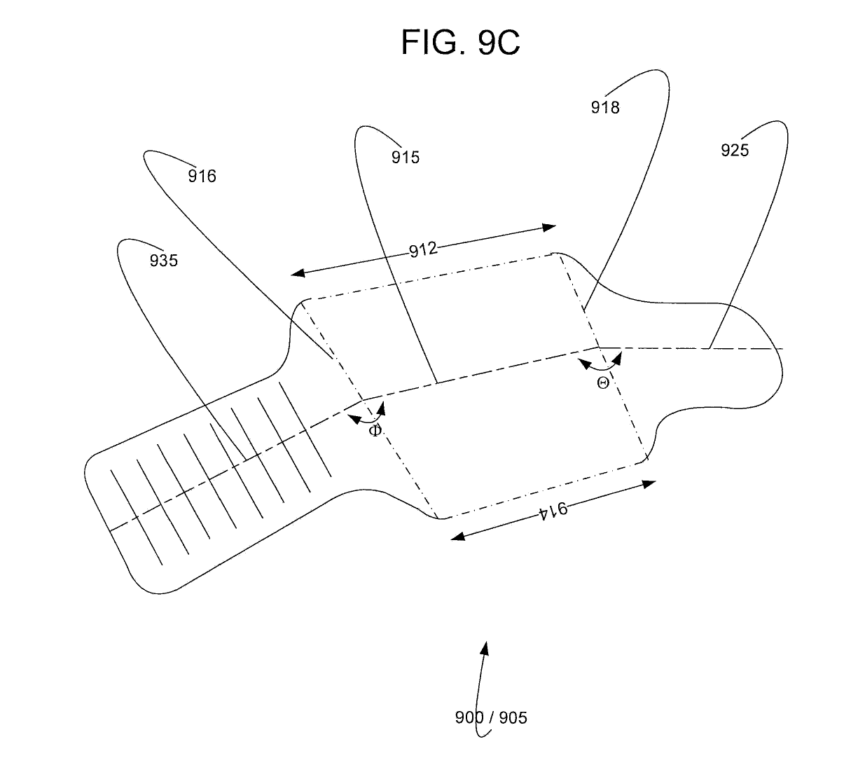

Referring now to FIG. 9A, Applicant's planar assembly 900 comprises three interconnected elements, namely a trapezoidal-shaped center portion 910, an insertion tab 920 extending outwardly from one side of center portion 910, and an insertion slot assembly 930 extending outwardly from an opposing side of center portion 920. In certain embodiments, Applicant's planar assembly 900 is formed from one or more of paper, cardboard, cloth, plastic, and combinations thereof.

In the illustrated embodiments of FIGS. 9A and 9C, insertion slot assembly 930 is formed to include a plurality of slots extending therethrough. More specifically, insertion slots 931, 932, 933, 934, 036, 037, 938, and 939, are oriented perpendicularly to longitudinal axis 935.

Referring once again to FIG. 9C, center portion comprises a first side having a length 912 and a second side having a length 914, wherein length 912 differs from length 914. In the illustrated embodiment of FIG. 9C, length 912 is greater than length 914.

Center portion 910 comprises a center portion longitudinal axis 915. Insertion tab portion 920 comprises an insertion tab longitudinal axis 925. Insertion slot assembly 930 comprises an insertion slot assembly longitudinal axis 935. Center portion longitudinal axis 915 is not coaxial with insertion tab longitudinal axis 925. Rather, center portion longitudinal axis 915 and insertion tab longitudinal axis 925 define an angle THETA. In certain embodiments, angle THETA is between about 10 degrees and about 20 degrees.

In addition, center portion longitudinal axis 915 is not coaxial with insertion slot assembly longitudinal axis 935. Rather, center portion longitudinal axis 915 and insertion slot assembly longitudinal axis 935 define an angle PHI. In certain embodiments, angle PHI is between about 10 degrees and about 20 degrees.

In certain embodiments, angle PHI does not equal angle THETA. In other embodiments, angle PHI equals angle THETA. In either event, insertion tab longitudinal axis 925 is not coaxial with insertion assembly longitudinal axis 935.

FIG. 9B illustrates embodiment 905 of Applicant's planar assembly, wherein trapezoidal-shaped center portion 915 is formed to include a plurality of apertures extending therethrough. These plurality of apertures facilitates cooling of the a patient's forearm. Planar assembly 905 comprises elements 920 and 930 as described hereinabove.

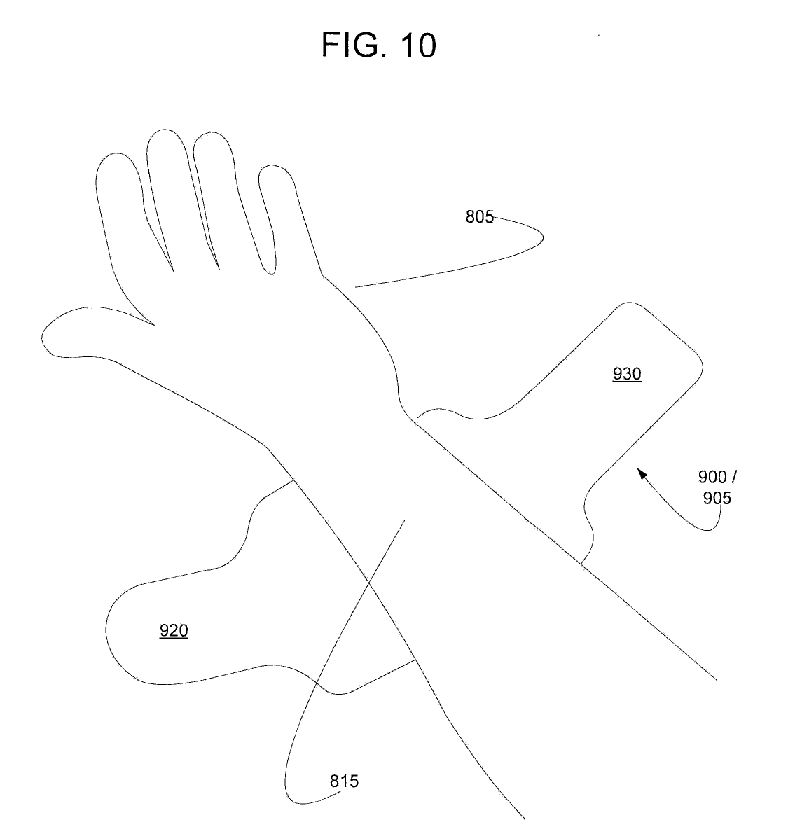

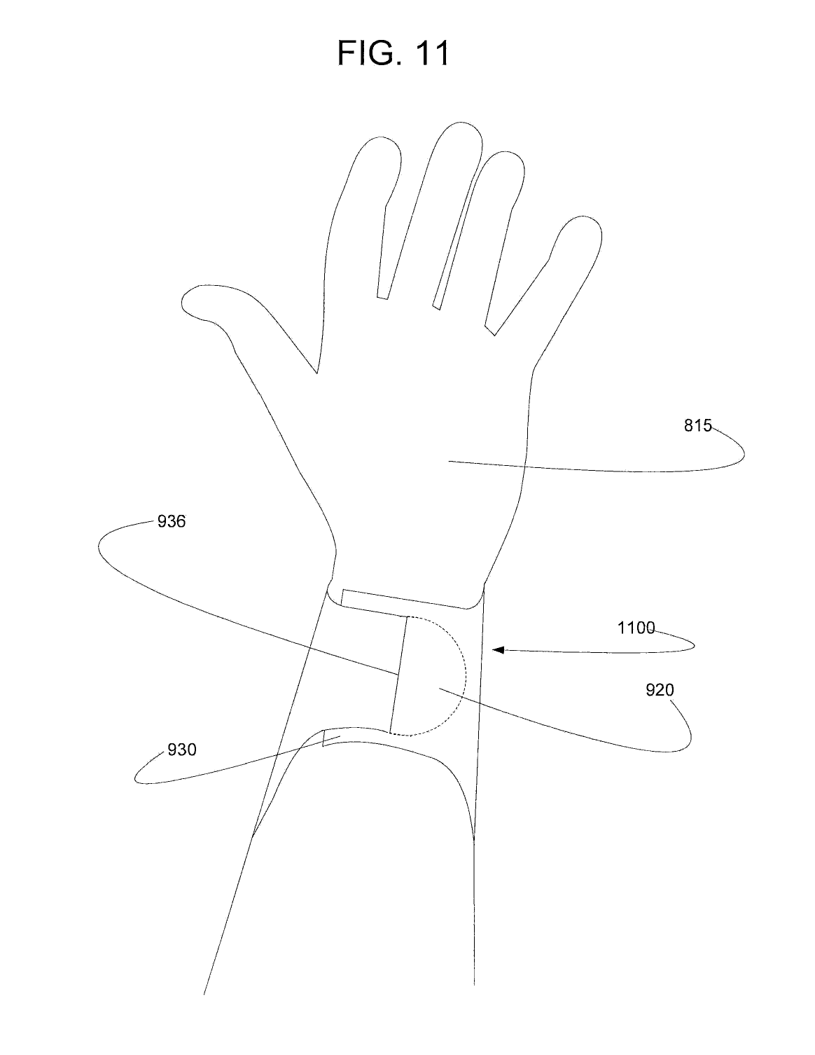

FIG. 10 shows Applicant's planar assembly 900/905 disposed beneath a patient's forearm 815. FIG. 11 shows Applicant's planar assembly 900 removeably disposed around a patient's forearm 815 to define intravenous line support sleeve 1100 having a truncated conical shape.

Referring now to FIGS. 10 and 11, the insertion slot assembly portion 930 of Applicant's planar assembly 900/905 is placed around the patient's forearm 815. Then the insertion tab portion 920 of Applicant's planar assembly 900/905 is placed over a distal end of insertion slot assembly 930, and a distal end of insertion tab 910 is inserted into an insertion slot. FIG. 11 illustrates insertion tab 920 having been inserted into insertion slot 936 to form Applicant's intravenous line support sleeve 1100.

In the illustrated embodiment of FIG. 11, intravenous catheter 820 has been advanced through the skin on the top of a patient's hand, and is removeably disposed in a vein disposed within a patient's hand 805. A portion 825 of that catheter remains external to hand 805. A first end 832 of intravenous line 830 is interconnected with catheter portion 825. A second end 835 of intravenous line 830 is interconnected with a fluid reservoir, wherein that fluid reservoir contains an infusible liquid formulation comprising a fluid medium, such as and without limitation water, saline, Ringer's Lactate, and the like, in optional combination with one or more medicaments disposed in the fluid medium. By “intravenous line,” Applicant means a flexible tubular member used to interconnect a fluid reservoir and an intravenous catheter.

Applicant's intravenous line support sleeve 1100 functions, inter alia, as a forearm protector. No portion of Applicant's intravenous line support sleeve 1100 is secured to, or attached in any way to, the patient's skin. In essence, intravenous line support sleeve 1100 “floats” on the forearm. Applicant's intravenous line support sleeve provides a substitute skin surface to one or more secure I.V. lines rather than attaching those line directed to the patient's often compromised skin.

Applicant's intravenous line support sleeve 1100 is not intended as a wound dressing. Rather, Applicant's intravenous line support sleeve 1100 comprises a free floating platform solely intended to be an attachment surface to which, for example and without limitation, securing tape is applied rather than attaching that tape to the patient's forearm. As those skilled in the art will appreciate, a patient's forearm may not tolerate repeated application of, and removal of, securing tapes without damage to the skin.

By “free floating platform,” Applicant means that intravenous line support sleeve 1100 is not attached to the patient's forearm 815 or hand 805 in any fashion. Quite to the contrary, when Applicant's intravenous line support sleeve 1100 is removeably disposed around a patient's forearm as illustrated in FIG. 11, rotation of patient's forearm does not result in any sort of associated movement of intravenous line support sleeve 1100. For example, FIG. 11 illustrates the patient's hand 805 with the palm in an upwardly-facing orientation, i.e. in an anterior-facing position. A pronational rotation of the patient's forearm, i.e. a rotation of the patient's forearm from the illustrated anterior-facing position to a posterior-facing position, with the palm now facing downwardly, does not result in any rotation of Applicant's intravenous line support sleeve 1100. Intravenous line support sleeve 1100 comprises the above-described “free floating” characteristic upon pronation regardless of whether intravenous line support sleeve 1100 is formed using planar assembly 900 or planar assembly 905.

Similarly, a supinational rotation of the patient's forearm, i.e. a rotation of the patient's forearm from a posterior-facing position to an anterior-facing position, with the palm now facing upwardly, does not result in any rotation of Applicant's intravenous line support sleeve 1100. Intravenous line support sleeve 1100 comprises the above-described “free floating” characteristic upon supination regardless of whether intravenous line support sleeve 1100 is formed using planar assembly 900 or planar assembly 905.

While the preferred embodiments of the present invention have been illustrated in detail, it should be apparent that modifications and adaptations to those embodiments may occur to one skilled in the art without departing from the scope of the present invention as set forth in the following claims.

I claim:

1. A planar assembly for use as an intravenous support sleeve, comprising:
a trapezoidal-shaped center portion;
an insertion tab extending outwardly from a first side of said center portion;
an insertion slot assembly extending outwardly from a second and opposing side of said center portion.

2. The planar assembly of claim 1, wherein:
said trapezoidal-shaped center portion comprises a first side having a first length and a second side having a second length;
said first length differs from said second length.

3. The planar assembly of claim 2, wherein:
said center portion comprises a center portion longitudinal axis;
said insertion tab comprises an insertion tab longitudinal axis;
said insertion assembly comprises an insertion assembly longitudinal axis;
said center portion longitudinal axis is not coaxial with said insertion tab longitudinal axis;
said center portion longitudinal axis is not coaxial with said insertion assembly longitudinal axis.

4. The planar assembly of claim 4, wherein:
said center portion longitudinal axis and said insertion tab longitudinal axis define a first angle;
said center portion longitudinal axis and said insertion assembly longitudinal axis define a second angle.

5. The planar assembly of claim 4, wherein:
said first angle is between about 10 degrees and about 20 degrees; and
said second angle is between about 10 degrees and about 20 degrees.

6. The planar assembly of claim 5, wherein said first angle differs from said second angle.

7. A method to secure an intravenous line, comprising:
supplying a planar assembly comprising a trapezoidal-shaped center portion, an insertion tab extending outwardly from a first side of said center portion, an insertion slot assembly extending outwardly from a second and opposing side of said center portion, wherein said insertion slot assembly is formed to include a plurality of slots extending therethrough;
wrapping said planar assembly around the patient's forearm;
inserting said insertion tab into one of said plurality of slots formed in said insertion slot assembly to form a tubular intravenous line support sleeve comprises the shape of a truncated cone, wherein said tubular intravenous line support sleeve comprises a free floating platform disposed around but not attached to the patient's forearm to be used as an attachment surface to which securing tape can be applied rather than attaching that securing tape to the patient's skin.

8. The method of claim 7, further comprising:
supplying an intravenous catheter, a fluid reservoir, and an intravenous line comprising a flexible tubular member;
removeably disposing said intravenous catheter in a vein disposed within a patient's hand;
interconnecting said intravenous catheter with said fluid reservoir using said intravenous line;
releaseably attaching said intravenous line to said tubular intravenous line support sleeve;
wherein no portion of said tubular intravenous line support sleeve is secured to or attached to the patient's skin.

9. The method of claim 7, wherein:
said trapezoidal-shaped center portion comprises a first side having a first length and a second side having a second length;
said first length differs from said second length.

10. The method of claim 9, wherein:
said center portion comprises a center portion longitudinal axis;
said insertion tab comprises an insertion tab longitudinal axis;
said insertion assembly comprises an insertion assembly longitudinal axis;
said center portion longitudinal axis is not coaxial with said insertion tab longitudinal axis;
said center portion longitudinal axis is not coaxial with said insertion assembly longitudinal axis.

11. The method of claim 10, wherein:
said center portion longitudinal axis and said insertion tab longitudinal axis define a first angle;
said center portion longitudinal axis and said insertion assembly longitudinal axis define a second angle.

12. The method of claim 11, wherein:
said first angle is between about 10 degrees and about 20 degrees; and
said second angle is between about 10 degrees and about 20 degrees.

13. The method of claim 12, wherein said first angle differs from said second angle.

14. The method of claim 7, wherein pronational rotation of said patient's forearm having said tubular intravenous line support sleeve disposed therearound does not cause rotation of said tubular intravenous line support sleeve.

15. The method of claim 13, wherein subinational rotation of said patient's forearm having said tubular intravenous line support sleeve disposed therearound does not cause rotation of said tubular intravenous line support sleeve.
